(19) United States
(12) Patent Application Publication
(10) Pub. No.: US 2023/0301786 A1
(43) Pub. Date: Sep. 28, 2023

(54) GRAFT COMPOSITE FOR HIP LABRUM RECONSTRUCTION

(71) Applicants: Smith & Nephew, Inc., Memphis,, TN (US); Smith & Nephew Orthopaedics AG, ZUG (CH); SMITH & NEPHEW ASIA PACIFIC PTE LIMITED

(72) Inventors: Michael Thyden, Billerica, MA (US); Stephen A. Santangelo, Tolland, CT (US)

(21) Appl. No.: 18/035,564

(22) PCT Filed: Nov. 3, 2021
(86) PCT No.: PCT/US2021/057921

§ 371 Date: May 5, 2023

Related U.S. Application Data
(60) Provisional application No. 63/110,434, filed on Nov. 6, 2020.

(51) Int. Cl. A61F 2/30; A61F 2/34

(52) U.S. Cl. CPC A61F 2/30756; A61F 2/34; A61F 2002/30108; A61F 2002/30461

(57) ABSTRACT

A composite graft is disclosed, that may replace a segment of the hip labrum during a labral tear reconstruction. The composite graft may include a first sheet that acts as a scaffold and a second sheet that comprises a biologically active component. The first and second sheet are formed in a tubular roll with a spiraled cross section, with alternating layers of the first and second sheet. The composite graft may provide structural integrity for the hip labrum supplemented with growth factors.

### CROSS-REFERENCE TO RELATED APPLICATIONS

This application claims benefit to and incorporates by reference in its entirety, U.S. Provisional Pat. No. 63/110,434, filed Nov. 6, 2020; titled “BIOLOGIC GRAFT AUGMENTATION FOR HIP LABRAL RECONSTRUCTION”.

### FIELD

The present disclosure relates to a graft composite for hip labrum reconstruction. The disclosure relates to a method of hip labrum repair with a graft composite and method of forming the graft composite.

### BACKGROUND

The hip labrum is a ring of tough cartilage-like tissue that encircles the hip’s socket, or acetabulum. The femoral head engages and slides along the hip labrum to facilitate the hip’s range of motion, allowing the thigh to rotate. The ring formation also acts like a seal and keeps synovial fluid, or joint fluid, within the hip joint capsule (synovial fluid reduces stress and friction and allows for smooth movement between the ball and socket). When the labrum is damaged, the hips range of motion, alignment, and synovial fluid can be affected.

Due to the high loads on the hip joint, and therefore hip labrum, labral tears may require reconstruction rather than repair thereof. This may require removal of the damaged ring segment and replacement with a graft. Alternatively, the graft may overlay the damaged or missing segment. This graft is configured to reconstruct the native anatomy and must withstand the loads from the hipjoint, facilitating hip range of motion, while also providing a dam to seal in the synovial fluid.

Grafts for the hip labrum may include allograft tissue to reconstruct the native anatomy. For example, fascia tissue such as the Fascia Lata or IT band may be provided in a sheet which is rolled into a tubular structure. The tubular structure may be rolled to a target diameter, that may be between 2-10 mm in diameter. The hip labrum varies both between patients, but also in location around the hip; hip labrums are typically wider and thinner at the front of the hip, and thicker at the back of the hip. Tube may then be trimmed to the appropriate length of the missing or prepared ring segment and inserted into the hipjoint. The tube may be attached at the site of the removed or missing labrum via a plurality of standard suture anchors. The allograft tissue is inert tissue and does not inherently have any growth factors and therefore does not promote any healing or reintegration process.

### SUMMARY

Disclosed herein is an improved graft, and method of formation that adds a material while rolling the tube. The additional material can include one that motivates biologic activity. In one aspect, there is disclosed an improved hip labrum graft, the graft defining a composite graft including both a scaffold component and a biologically active component. The biologically active component is configured to motivate or increase biologic activity between the graft and adjacent tissue. Scaffold component and biologically active component may be provided as separate components and combined during the procedure. Also disclosed herein is a method of forming a composite graft and a method of reconstructing a hip labrum with the composite graft.

Composite graft may be provided as at least two separate components. One component may be a sheet that acts primarily as a scaffold component, the other component may be a sheet that acts primarily as a biologically active component. Each sheet may be separately formed and different from one another. The two components may be wound around each other to form a tubular graft with a spiral cross section including alternating layers of each component. The scaffold component is configured to provide the structural integrity for the hip labrum, while the biologically active component is configured to promote healing and reintegration. The scaffold component may control the rate of exposure of the biologically active sheet to the adjacent tissue.

A first example composite graft for reconstruction of the hip labrum may include two sheets. A first sheet may include a scaffold component while a second sheet may include a biologically active component. The first and second sheets can be rolled together to form the composite graft into a tube with a spiraled cross section, defining alternating layers of the first and second sheets. The first sheet defines the outer-most circumferential surface of the tube and can be configured to be in direct contact with a tissue surface of a subject (e.g., human subject). This protects the more fragile biologically active component during manipulation of the composite graft and controls the rate of interaction between the biologically active component and adjacent tissues of the subject.

In some particular embodiments, the composite graft is sized to replace a deficiency of the hip labrum. The first sheet is configured to deliver the second sheet to the labrum and control the exposure of the biologically active component to the hip labrum. The first sheet may delay the exposure of the biologically active component. The scaffold component may be an autologously derived tissue, an allograft derived tissue, a xenograft derived tissue, or a synthetic material, or any combination thereof. The biologically active component may include growth factors. The biologic active component may include placental or umbilical tissues. The first and second sheet may be arranged such that the second sheet is absent at a center portion of the tube. The first and second sheet may be rolled together to form at least two complete revolutions around an axis of the tube.

An example method of replacing a segment of a hip labrum is also disclosed. The method may include forming a composite graft that includes a scaffold component formed as a first sheet and a biologically active component formed as a second sheet. The second sheet may be placed onto the first sheet and the two sheets rolled to form the composite graft into a tube with a spiral cross section defining alternating layers of the first and second sheet. The first sheet defines an outermost surface of the tube. The tube is then secured along a prepared segment length of the hip labrum.

In some example methods, the first sheet may include an autologously derived tissue, an allograft derived tissue, a xenograft derived tissue, or a synthetic material, or any combination thereof. The first sheet may include allograft derived tissue and the second sheet may include placental tissue. The second sheet may include placental or umbilical tissues. The method may include arranging the second sheet onto the first sheet so that rolling further comprises first rolling the first sheet around itself before rolling the first and second sheet together, and thereby forming a tube with a central portion absent the second sheet. The method may include arranging the second sheet onto the first sheet so that rolling comprises rolling the second sheet around itself before rolling the first and second sheet together, and thereby forming a tube with a central portion absent the first sheet. The method may include arranging the second sheet such that rolling the first and second sheet exposes biologically active component at both end surfaces of the tube.

Another example composite graft embodiment is disclosed, including a first sheet comprising fascia tissue and a second sheet comprising placental tissue. The first and second sheets are rolled together to form the composite graft into a tube or rod with a spiraled cross section. The spiraled cross section defines a plurality of alternating layers of the first and second sheet, with the first sheet defining an outermost layer of the plurality of alternating layers.

In some example methods, the composite graft replaces a segment of the hip labrum. The first and second sheets may be arranged such that the second sheet is absent at a central portion of the tube. The first and second sheets may be rolled around each other defining at least two complete revolutions around an axis of the tube. The tube defines an elongate body with opposing end surfaces, and the second sheet may be exposed at both of the opposing end surfaces.

An example method of adding a biological active component to a graft is disclosed, including disposing a layer of the biologically active component on a planar external surface of a tissue graft. The tissue graft is then rolled into a tubular configuration with the biologically active component disposed thereon. The tubular configuration includes a spiral cross section, with alternating layers of the tissue graft and biologically active component. The rolling is in a direction such that the tissue graft is an external-most layer of the alternating layers. The graft with the biologically active component included is then securing the tissue graft to the target graft site.

The composite graft can have a rolled shape. An aspect ratio of the length (L) to the height (H) of the rolled composite can be modified as desired. In certain embodiments, the aspect ratio (L:H) can be less than one, one, or greater than one. By way of example, non-limiting aspect ratios (L:H) can be 0.1 to 1, 0.5 to 1, 1 to 10, 1 to 5, 1 to 2, 1 to 1.5, etc.).

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The disclosure will be more fully understood by reference to the detailed description, in conjunction with the following figures, wherein:

FIG. 1A illustrates an example of a composite graft in accordance with this disclosure;

FIG. 1B illustrates a cross section of the composite graft in accordance with this disclosure;

FIG. 1C illustrates a segment of the composite graft in accordance with this disclosure;

FIG. 2 illustrates components of the composite graft in accordance with this disclosure;

FIG. 3 illustrates an example arrangement of the components of the composite graft before rolling, in accordance with this disclosure;

FIG. 4A illustrates another example arrangement of the components of the composite graft before rolling, in accordance with this disclosure;

FIG. 4B illustrates a resulting cross section of the composite graft with the arrangement illustrated in FIG. 4A, in accordance with this disclosure;

FIG. 5A illustrates another example arrangement of the components of the composite graft before rolling, in accordance with this disclosure;

FIG. 5B illustrates a resulting cross section of the composite graft with the arrangement illustrated in FIG. 5A in accordance with this disclosure; and

FIGS. 6A-6C illustrates a method of reconstructing the hip labrum with a composite graft.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” “have” and/or plural forms of each are open ended and include the listed parts and can include additional parts that are not listed. “And/or” is open-ended and includes one or more of the listed parts and combinations of the listed parts. Use of the terms “upper,” “lower,” “upwards,” and the like is intended only to help in the clear description of the present disclosure and are not intended to limit the structure, positioning and/or operation of the disclosure in any manner.

Disclosed herein is a composite graft, including a scaffold component configured to provide the structural integrity for the hip labrum segment. A biologically active component is added to the scaffold component, the biological active component configured to motivate biologic activity between the graft and adjacent tissues. Biological active component may add growth factors, to promote a healing response. The biologically active component may promote reintegration of the composite graft with the adjacent tissue. The graft composite may be formed during the surgical procedure, each component individually provided and combined during graft preparation. For example, each component may be provided in a sheet, that may be layered one on top of the other before rolling into a tube. This may form a tube with a cross section similar to a cinnamon roll. Tube preferably defines a solid cross section, with minimal voids. Tube may also be called a tubular rod.

### Biologically Active Component

The biologically active component can be a component that motivates biologic activity between the adjacent tissues and graft. It may include at least one biologic material and may include a combination of different active components. Biologically active component may include growth factors, that may be live. The biologically active component may improve integration between the graft and adjacent tissues. The biologically active component may be provided as a sheet or strip and may preferably be flexible so that it can be rolled into a tube. It may provide a non-structural portion of the graft and may be fragile, requiring protection or structural support. This is provided by the scaffold component. Stated in another way, the biologically active component alone may provide insufficient or negligible structural integrity as a reconstruction graft for the hip labrum. The biologically active component is configured to react upon exposure to adjacent biological tissues and support tissue healing and integration between the graft and adjacent tissues. The release rate or timing of this biological component may be controlled by being at least partially covered by the scaffold material thus avoiding rapid breakdown once in-vivo.

The biologically active component may include any biologic material (e.g., whole tissue or tissue-derived material) with the properties described herein. The biologically active component can be an autograft, allograft, or xenograft, or any combination thereof. Non-limiting examples of such biologic materials include biomatrices, such as acellular or extracellular tissue matrices. Non-limiting examples may include placental or umbilical tissue such as for example Grafix or Stravix, both offered for sale by Smith and Nephew. Grafix includes a cryopreserved placental membrane comprised of an extracellular matrix (ECM) rich in collagen, growth factors, fibroblasts, mesenchymal stem cells (MSCs), and epithelial cells native to the tissue. Stravix includes a cryopreserved human placental tissue composed of umbilical amnion and Wharton’s jelly. Stravix retains the native collagen and hyaluronic acid-rich extracellular matrix (ECM), endogenous growth factors, and endogenous cells including epithelial cells, fibroblasts, and mesenchymal stem cells (MSCs) found in placental tissue. The biologically active component may be a placental product comprising an immunocompatible chorionic membrane. Such placental products can be cryopreserved and contain viable therapeutic cells after thawing. Example placental products are disclosed in more detail in at least U.S. Pat. 10,265,344, and 10,258,650 commonly owned and herein disclosed in its entirety by reference. The biologically active component may comprise a previously cryopreserved umbilical tissue, wherein after cryopreservation and subsequent thawing the umbilical tissue comprises any one of, any combination of, or all of: a) viable cells native to the umbilical tissue; b) tissue integrity of native umbilical tissue; c) one or more growth factors that are native to the umbilical tissue; and d) depleted amounts of one or more types of functional immunogenic cells. Example Umbilical products are disclosed in more detail in at least U.S. Pat. 10,624,931 commonly owned and herein disclosed in its entirety by reference. The biologically active component may comprise a non-homogenized chorionic matrix, a homogenized amniotic matrix, and a homogenized UC (UC) matrix, wherein the non-homogenized chorionic matrix comprises viable cells. Example products are disclosed in more detail in at least U.S. Pat. Application 2017/0368105 commonly owned and herein disclosed in its entirety by reference.

Other non-limiting examples may include a biologically active component crosslinked with a polymer. Other non-limiting examples may be in a spreadable form and may include platelet-rich plasma (PRP), B-mac (bone marrow aspirate), fibrin blood clot, or minced adipose tissue, or any combination thereof. These may be spread on the scaffold component, before rolling/tubularizing the graft.

### Scaffold Component

Scaffold material may be provided as a sheet or strip that is flexible and can be rolled into a tube with a spiral cross section. Tube includes material throughout its cross section and may be called a tubular rod. Scaffold component may provide substantially all of the structural integrity of the graft. As a hip labrum graft, the scaffold provides the structural properties to engage and support the hip joint and seal the synovial fluid therein. Stated another way, the addition of the biologically active component may not add significant structural integrity to the graft, although some alteration to the structural integrity may occur. Scaffold component can also protect the biological active component which may be fragile. Scaffold component is configured to provide structural support and slow down or control the breakdown rate of the biologically active component once in-vivo. Scaffold component may be autologously derived tissue, allograft derived tissue, xenograft derived tissue or synthetic material, or any combination thereof. Scaffold component may be a mix of xenograft and synthetic material. The synthetic may be a collagen/polymer mix. The scaffold could include a collagen (bovine/porcine) reconstitution or hybridized material (mixing fast absorbing with longer term or non-absorbable). Synthetic materials may include resorbable magnesium, PLLA, PLTA, or polycitric acid. The scaffold may be made entirely from at least one synthetic material.

The scaffold may be a natural tissue, provided, prepared as a sheet. For example allografts in the form of the Fascia Lata or iliotibial (IT) band have been used as a graft for hip labrum repair, as they offer an improved biologic foundation, structural (cellular/fibril) organization, longevity (low degradation rate) and a match in flexibility (to conform to the geometry of the acetabulum). These tissues all support the demands of the repair.

Scaffold component may include a mix of absorbable and non-absorbable biocompatible materials. Utilizing bioabsorbable materials may allow for increased avenues over time for the adjacent tissues to interact with the biologically active component. In other non-limiting examples, scaffold may be provided with some degree of porosity as provided or alternatively porosity added during preparation of the graft during the procedure. This porosity may provide conduits for cell penetration between the biologically active materials and adjacent tissues without significant loss in structural integrity, required for the hip labrum to function.

In some embodiments, a suitable bioabsorbable material for use in the present disclosure is made of a poly-hydroxybutyrate (a polyhydroxyalkanoate), such as the TephaFlex polymer produced by Tepha, Inc. of Cambridge, Mass. In some embodiments, useful bioabsorbable materials include, for example, polylactic acid (PLA), polyglycolic acid (PGA), polylactideglycolide acid (PLGA), polydioxanone (PDO), or polycaprolactone (PCL), or any combination thereof. In some embodiments, bioabsorbable materials suitable for use in the present disclosure include polyanhydrides, polyorthoesters, poly(amino acids), polypeptides, polydepsipeptides, nylon-2/nylon-6coplyamides, poly(alkylene succinates), poly(hydroxyl butyrate) (PHB), poly (butylene diglocolate), poly (C-caprolactone), polydihydropyrans, polyphosphazenes, poly (ortho ester), poly (cyano acrylates), modified polysaccharides, cellulose, starch, chitin, modified proteins, collagen, fibrin, and combinations and copolymers thereof.

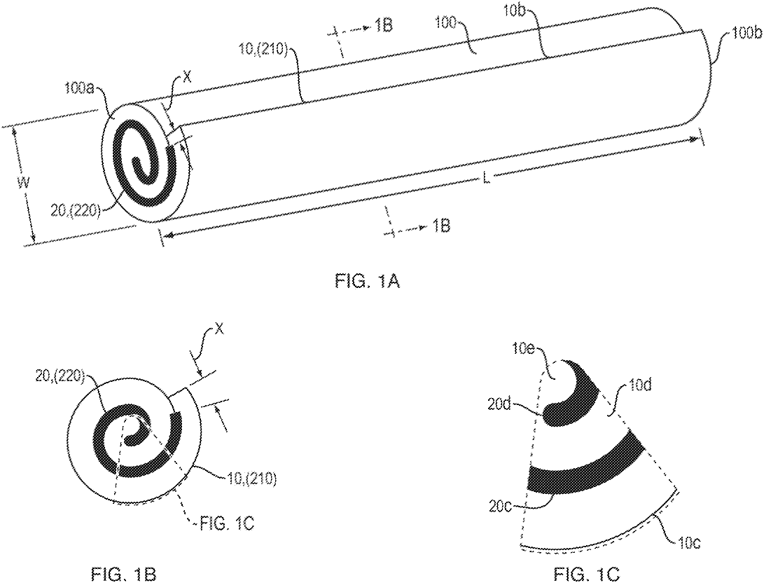

FIG. 1A illustrates a first example composite graft 100 formed in a tubular rod shape with a spiral cross section defining a plurality of layers of a scaffold component 10 alternating with layers that include a biologically active component 20. Once in this tubular shape, the edge 10b may be fixed in place using fixation means known in the art. For example, a whipstitch may be threaded through portions of the graft 100 (not shown). Suture may be looped circumferentially around the graft 100 and tied in place (not shown). A layer of adhesive may be placed long edge 10b (not shown). A first example cross section of the two components 10, 20 may be seen exposed at end 100a, and in cross section in FIG. 1B. These spiraled layers may extend along the entire length L of composite graft 100, such that the cross section of layers remains unchanged therealong. Tubular rod may be formed to have a diameter between 2-10 mm and between 2-14 cm. Outer surface of graft 100 is preferably formed entirely of the scaffold material 10. This may protect the more fragile biologically active material 20. As such scaffold component 10 may extend further than biologically active component 20, by a distance X to ensure that the biologically active component 20 is covered and protected.

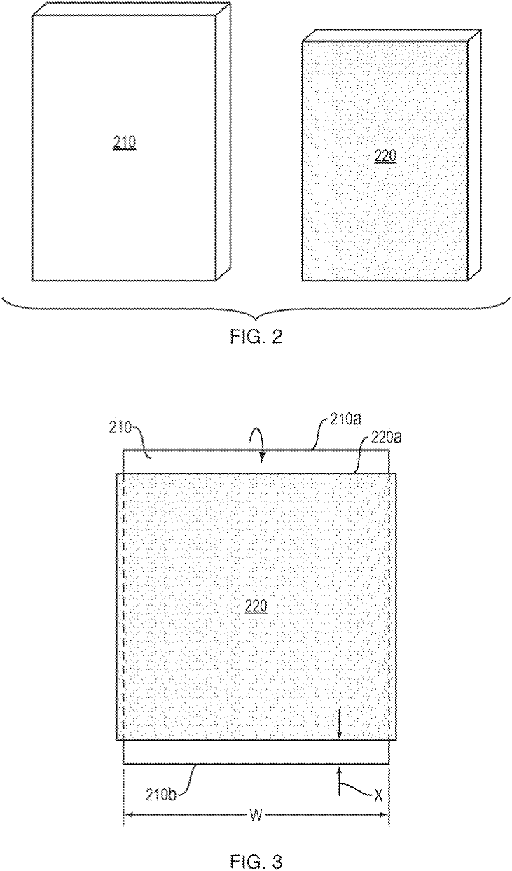

FIG. 2 illustrates an example method of forming composite graft 100, including a first sheet defining a scaffold sheet 210 and a second sheet defining a biologically active component sheet 220. Scaffold sheet 210 may have a length up to 20 cm, a width up to 20 cm and a thickness between 0.5 mm-5 mm. Scaffold dimensions are generally configured to provide the structural integrity to the graft. Scaffold thickness is also configured to expose the biologically active component after some digestion and remodeling of the scaffold component during integration between the composite graft and local tissues. Therefore, a thinner sheet 220 may expose the biologically active component faster. A plurality of layers formed by the spiral cross section may expose portions of the biologically active layer over a longer period of time, or at controlled time lapses as each layer of spiral breaks down and assimilates with the adjacent tissues. As the body incorporates the composite graft 100 and integrates it, the biologically active component 20 may be exposed and activated. The spiraled formation is preferable, as it forms layers of the biologically active component 20 between the scaffold 10 that may control its release in a more controlled manner, over a preferred length of time while the scaffold 10 is broken down. An outer-most circumferential surface of the tubular composite graft is preferably the scaffold component 10 to protect the biologically active component 20 on insertion and limit immediate or acute breakdown of the biologically active component.

For example, turning to FIG. 1C illustrating a segment of the graft 100, assuming that the graft 10 is remodeled from the external surfaces and in, and in a simplified form of the tissue integration mechanism, the scaffold layer segment 10C is first partially digested and remodeled, exposing layer segment 20C of the biologically active component 20. The period of time before exposure is defined at least in part by the (radial) thickness of segment 20C. Once biologically active component 20C is exposed, it may react with local tissues, introduce growth factors around the graft and disintegrate, while being supported by at least scaffold segment 10D. Segment 10D may then follow the mechanism of segment 10C to eventually expose biologically active segment 20D and so on, depending on the number of revolutions of the spiral. Composite graft 100 may include a plurality of each sheet (210, 220) layered on top of each other. Each component may be provided in a standard sheet length and width and may be trimmed during the procedure.

In a first example method, illustrated in FIG. 3, a biologically active component sheet 220 may be placed on top of a scaffold sheet 210. The biologically active component sheet 220 may be a different footprint than the sheet 210. This may define an offset X on at least one side. Biologically active component sheet 220 may be of equivalent width “W” to scaffold sheet 210, to expose at least some of the biologically active material acutely, at the open ends 100a, 100b. Alternatively, the tubular graft 100 may be trimmed to length (L), which may expose the biologically active material at ends 100a, 100b. Edge 210a of scaffold sheet 210 may be rolled, gradually incorporating edge 220a as the rolling continues. This may form a composite graft 100 with a scaffold sheet 210 extending beyond the biologically active component sheet 220 by distance X. In other methods, the sheet 220 may be placed on sheet 210 and rolled to a target diameter of tubular rod, and then the sheets 210, 220 trimmed. Biologically active component sheet 220 may be trimmed to form covered length X.

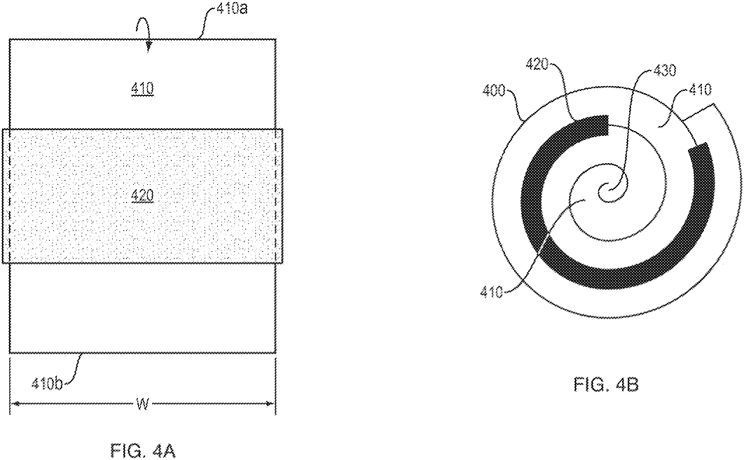

FIGS. 4A and 4B illustrates another composite graft 400 and method of formation. In this embodiment, composite graft 400 may form a central or core portion that is absent biologically active component 20. FIG. 4A illustrates a biologically active component sheet 420 that is initially laid on top of scaffold sheet 410, and spaced from both edges 410a, 410b. FIG. 4B illustrates an end view (or cross section) of tubularized composite graft 400, with the biologically active sheet 420 absent both at the outer surface of the graft 400 and also absent a central core portion 430 of the graft 400. The scaffold sheet 410 may wind around itself for a complete revolution (360 degrees) around an axis of the graft 400 before the biologically active sheet 420 is incorporated.

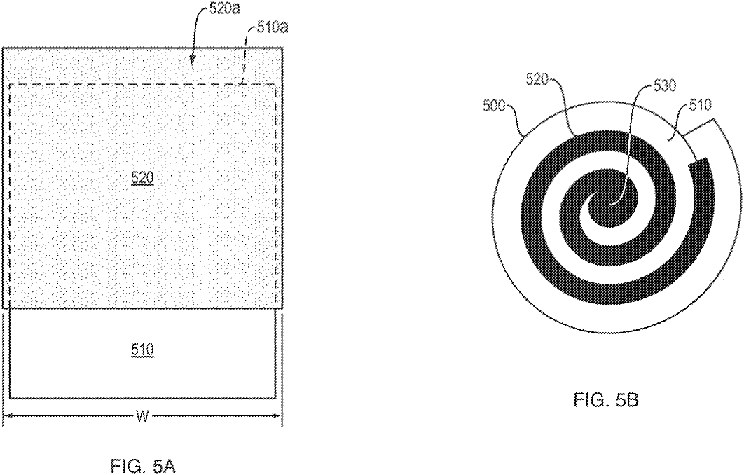

FIGS. 5A and 5B illustrates another method of forming a composite graft 500, the graft 500 having a central portion 530 absent the scaffold component 510. This embodiment may provide a larger conduit for cell migration into the graft 500 along the center of the tubular graft 500. FIG. 5A illustrates the biologically active component sheet 520 that is initially laid on top of scaffold sheet 510 and extends over edge 510a of scaffold 510. FIG. 5B illustrates an end view (or cross section) of tubularized graft 500, with the scaffold component 510 absent along a central portion 530 of the graft. Scaffold component 510 may define the external most surface of composite graft 500. The biologically active component 520 may wind around itself for a complete revolution (360 degrees) of the spiral before the scaffold sheet 510 is incorporated.

In other embodiments, the scaffold and biologic source may be wound around each other to form a non-spiral shape. For example, the composite graft could be formed by snaking or zig zagging the two layers back and forth and then suturing them in place. Also multiple layers of each material could be used. For example, the biologically active component could be initially sandwiched between two layers of scaffold components before rolling or winding them together.

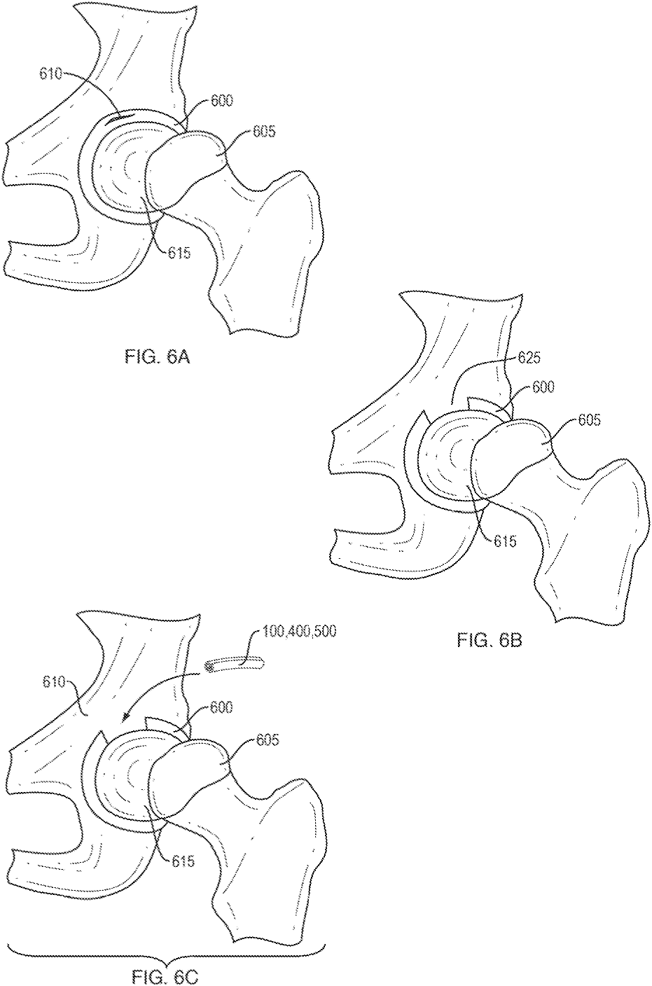

A method of hip labrum reconstruction is illustrated in FIGS. 6A-6C. FIG. 6A illustrates a hip joint, including a hip labrum 600 with an example labral tear 610. Femur 605 has been partly moved for ease of understanding of the procedure. Labrum 600 extends around the rim of an acetabular cup 615. The method may include removing a segment of the hip labrum 600 that has a tear 610 (FIG. 6B), exposing a surface of acetabular rim 625. In some methods, the entire hip labrum may be removed. The tissues around the removed segment of the hip labrum may then be prepared. For example, the tissue surfaces, including the exposed acetabular rim may be debrided and cleaned. Anchor pilot holes may be prepared within the acetabular bone (not shown). The segmental length of the removed segment may be measured. The cross-sectional width of the labrum immediately adjacent the removed segment may also be measured. This provides the target diameter and length of the composite graft (100, 400, 500). A composite graft (100, 400, 500) may then be trimmed to size and secured to the acetabular bone 615 along the removed segment (FIG. 6C). Securing the composite graft (100, 400, 500) may include anchoring the composite graft (100, 400, 500) to the acetabular bone at several locations along the removed segment. Anchoring may include placing suture anchors within the prepared pilot holes. The composite graft (100, 400, 500) may be provided preformed. The composite graft (100, 400, 500) may include autograft derived tissues, and therefore the method may include harvesting and preparing the autograft derived tissues. The composite graft (100, 400, 500) may be provided in a disassembled form, such as in a first sheet of scaffold material and a second sheet of a biologically active material. The method may include arranging the two sheets and rolling them together, to form a tubular rod with a spiral cross section including layers of both sheets. Suture may stitch composite graft (100, 400, 500) in the tubular form. The first sheet may define the outermost layer of the tube and thereby the composite graft (100, 400, 500). The first sheet may be fascia tissue, with minimal viable cells, provided as an allograft. The first sheet may include some porosity to enable cell migration therethrough. The second sheet may be placental tissue, including growth factors. The second sheet may be fragile and therefore is carried by the first sheet.

In other methods, the composite graft may be provided in a disassembled form, including a first sheet of scaffold material and a second material including a biologically active material that may be a gel or spreadable consistency. The method may include spreading the biologically active material over the first sheet and then rolling the first sheet, to form a tube with a spiral cross section including layers of the first sheet with the biologically active material in between each layer. The first sheet may define the outermost layer of the tube and thereby the composite graft. The first sheet may be fascia tissue, with minimal viable cells, provided as an allograft. The first sheet may include some porosity to enable cell migration therethrough. The biologically active material may be provided in a gel form and may include growth factors.

The composite graft can be packaged into a container. In some aspects, the composite graft can be pre-rolled and ready for use by removing the composite graft from the container and optionally sized to a desired length. In some aspects, the composite graft can be packaged in a disassembled form such that the scaffold material is included in a first compartment within the container, and the biologically active material is in a second compartment within the container. The container can be made of a variety of materials, non-limiting examples of which can include plastic, metal (e.g., aluminum), or combinations thereof. The container can also include instructions on how to form and/or use the composite graft. In certain aspects, the container can be vacuum sealed. In some aspects, the composite graft can be sterilized.

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

What is claimed is:

1. A hip labrum graft comprising:
a first sheet comprising a scaffold component; and
a second sheet comprising a biologically active component,
wherein the first and second sheet are rolled together to form the hip labrum graft into a tubular rod with a spiraled cross section, defining alternating layers of the first and second sheet, and
wherein the first sheet defines an outer-most circumferential surface of the tubular rod.

2. The hip labrum graft of claim 1 wherein the graft is sized to replace a deficiency of the hip labrum.

3. The hip labrum graft of claim 1 wherein the first sheet is configured to deliver the second sheet to removed portion of labrum and control exposure of the biologically active component to the tissues adjacent the removed portion of labrum.

4. The hip labrum graft of claim 1 wherein the scaffold component is an autologously derived tissue, an allograft derived tissue, a xenograft derived tissue or a synthetic material.

5. The hip labrum graft of claim 1 wherein the biologically active component includes growth factors.

6. The hip labrum graft of claim 1 wherein the biologically active component includes placental or umbilical tissues.

7. The hip labrum graft of claim 1, wherein the first sheet and second sheet are arranged such that the second sheet is absent at a center portion of the tubular rod.

8. The hip labrum graft of claim 1 wherein the first and second sheet are rolled together defining at least two complete revolutions around an axis of the tubular rod.

9-16. (canceled)

17. A composite graft comprising:
a first sheet comprising fascia tissue; and
a second sheet comprising placental tissue;
wherein the first and second sheet are rolled together to form the composite graft into a tube with a spiraled cross section, defining alternating layers of the first and second sheet, with the first sheet defining an outermost layer of the alternating layers.

18. The composite graft of claim 17 wherein the composite graft is configured to replace a segment of the hip labrum.

19. The composite graft of claim 17, wherein the first and second sheets are arranged such that the second sheet is absent at a central portion of the tube.

20. The composite graft of claim 17 wherein the first and second sheets are rolled together defining at least two complete revolutions around an axis of the tube.

21. The composite graft of claim 17 wherein the tube defines an elongate body with opposing end surfaces, and wherein the second sheet is exposed at both of the opposing end surfaces.

22. A method of adding a biologically active component to a tissue graft comprising:
disposing a layer of the biologically active component on a planar surface of a tissue graft;
rolling the tissue graft with the biologically active component disposed thereon into a tube with a spiral cross section, with alternating layers of the tissue graft and biologically active component, the rolling in a direction that defines the tissue graft as an external-most layer of the alternating layers.

23. The method of claim 22 wherein the tissue graft includes an autologously derived tissue, an allograft derived tissue, a xenograft derived tissue or a synthetic material.

24. The method of claim 22 wherein the tissue graft includes allograft derived tissue and wherein the biologically active component includes placental tissue.

25. The method of claim 22 wherein the biologically active component includes is placental or umbilical tissues.

26. The method of claim 22 wherein the biologically active component is spread over a planar surface of the tissue graft.

27. The method of claim 22 further comprising arranging the biologically active component onto the tissue graft so that rolling first rolls the tissue graft around itself before adding the biologically active component, and thereby forming a composite graft with a central portion absent the biologically active component.

28. The method of claim 22 further comprising arranging the biologically active component onto the tissue graft so that rolling comprises first rolling the biologically active component around itself before adding the tissue graft, and thereby forming a composite graft with a central portion absent the tissue graft.

29. The method of claim 22 further comprising arranging the biologically active component such that rolling exposes the biologically active component at both ends surfaces of the tube.
